(19) United States
(12) Patent Application Publication
(10) Pub. No.: US 2016/0153105 A1
(43) Pub. Date: Jun. 2, 2016

(54) PRODUCING A NANOPORE FOR SEQUENCING A BIOPOLYMER

(71) Applicant: SIEMENS AKTIENGESELLSCHAFT, München (DE)

(72) Inventor: Walter Gumbercht, Herzogenaurach (DE)

(21) Appl. No.: 14/905,570

(22) PCT Filed: Jul. 9, 2014
(86) PCT No.: PCT/EP2014/064719

§ 371 Date: Jan. 15, 2016

(30) Foreign Application Priority Data
Jul. 23, 2013 (DE) 102013214341.9

(51) Int. Cl. C25D 5/02; G01N 27/447; C12Q 1/68

(52) U.S. Cl. CPC C25D 5/02; C12Q 1/6869; G01N 27/44791

(57) ABSTRACT

A process for producing at least one nanopore with a predetermined diameter for sequencing a biopolymer is provided herein. This process includes providing at least one electrode and at least two nanoparticles in an intervening space between the electrode and a delimiting component opposite to the electrode. The electrode is coated with an electrically conductive material with resultant mechanical fixing of the at least two nanoparticles in the intervening space, thus producing a fixed porous arrangement. Charging of the electrically conductive material and/or another electrically conductive material to the fixed porous arrangement permits the establishment of a predetermined diameter of at least one pore, such as the formation of the nanopore. A process for sequencing the biopolymer with the aid of a fixed porous arrangement, as well as a corresponding device, are also provided herein.

The present patent document is a §371 nationalization of PCT Application Serial Number PCT/EP2014/064719, filed Jul. 9, 2014, designating the United States, which is hereby incorporated by reference, and this patent document also claims the benefit of DE 10 2013 214 341.9, filed on Jul. 23, 2013, which is also hereby incorporated by reference.

### TECHNICAL FIELD

The embodiments relate to a method for producing a nanopore for sequencing a biopolymer, such as a nucleic acid or a protein.

### BACKGROUND

In the case of sequencing biopolymers by nanopores, e.g., a nucleic acid (such as a DNA, RNA, or an oligonucleotide) passes through a biological or artificial nanopore. In the case of sequencing, e.g., nucleic acids, individual bases of the nucleic acid strand may be analyzed in this case as a result of a change in the ion conductivity in the pore (electrical pore resistance) when the nucleic acid passes through the nanopore. In this case, a sample of the nucleic acid is passed through the nanopore via an electric field, e.g., by electrophoresis. When different nucleotides pass through the nanopore, the ion current changes. This change is dependent on the nucleotide that passes through the pore, such that the nucleotide may be detected and the sequence of the nucleic acid may be determined.

Alternatively, it is possible to measure a tunneling current in the nanopore, transversely with respect to the transport direction of the biopolymer, as the biopolymer passes through, the magnitude of the tunneling current being dependent on, e.g., the nucleotide or the amino acid situated in the nanopore. This method of “transverse tunneling nanopore” sequencing is a promising method for sequence determination with a higher resolution. The principle of “transverse tunneling nanopore” sequencing is described in the U.S. Pat. No. 6,627,067 B1.

In order to achieve a high reliability of nucleic acid sequencing by tunneling current analysis, it is desirable to produce nanopores having a small pore diameter, (e.g., between 1 and 5 nanometers or between 1 and 2 nanometers), which are contacted by two electrodes.

To produce nanopores, Ayub et al. (Journal of Physiology, Condens, Matter 22 (2010) 454128) use a uniform metal layer that is reduced in size by electrolytic metal deposition. By this, with high outlay, a maximum of one nanopore may be produced. By this, it is only possible to produce nanopores suitable for use of pore resistance measurement, but not for tunneling current measurement, which may require pores with two electrodes in each case.

Tsutsui et al. (Nature Nanotechnology, April 5, 286-290, 2010) report so-called “nanofabricated mechanically controllable break junctions” (“nano-MCBJs”), a method that may be used to generate gaps of one nanometer that, although they are very narrow, are laterally very wide, for example. As such, a metrological resolution of bases is not possible. In addition, nanoelectrode junctions are reported, which enable small spacings of the electrodes approximately in “punctiform fashion”. This does not provide that the nucleic acid strand to be sequenced is guided in the junction. It may “drift away laterally” and thus leave the junction, and the measurement signal is lost.

DE 10 2012 21 76 03.9 describes a method for producing nanopores for tunneling current analysis, wherein a mixture of electrically conducive and nonconductive nanoparticles is arranged between two electrodes. As a result, interspaces between two nanoparticles may shape a nanopore. The probability of such an arrangement forming is very low, however, and so this method may be employed only to a limited extent.

The production of nanopores with tunnel electrode arrangements has hitherto been realized by expensive and time-intensive nanostructuring methods, or by technically unsuitable methods.

### SUMMARY AND DESCRIPTION

The scope of the present invention is defined solely by the appended claims and is not affected to any degree by the statements within this summary. The present embodiments may obviate one or more of the drawbacks or limitations in the related art.

An object achieved by the embodiments is an increase in the efficiency of production and utilization of tunneling current nanopores.

The method for producing at least one nanopore having a predetermined diameter for sequencing a biopolymer is based on the concept of coating at least one electrode with a conductive material and additionally narrowing at least one pore of a resultant fixed porous arrangement by coating, for example. A predetermined diameter may thus be established.

The method is carried out in a chamber of an apparatus. The method includes providing at least one electrode and at least two nanoparticles in the chamber, wherein the at least two nanoparticles are arranged in an interspace between the electrode and a delimiting component situated opposite the electrode; and coating at least the electrode with an electrically conductive material and thereby mechanically fixing at least one of the nanoparticles in the interspace, such that a fixed porous arrangement arises in the interspace.

A fixed porous arrangement is an arrangement of nanoparticles in which at least a portion of the nanoparticles is fixed by an electrically conductive material on the electrode and/or the delimiting component, and which includes a network of pores. As a result of the coating, e.g., electrically conductive nanoparticles may be brought into electrical contact with one another. In addition, the coating enables the formation of a nanopore by the nanoparticles as delimitation.

In this case, a nanoparticle is an assemblage of from a few to a few thousand atoms or molecules, the diameter of which may be between 1 and 100 nanometers. The use of, e.g., only a plurality of nonconductive nanoparticles simplifies the method since there is no need to provide nanoparticles of different materials. The terms “nonconductive” and “conductive” are used hereinafter in the sense of “nonelectrically conductive” and “electrically conductive,” respectively.

The method is characterized by filling the fixed porous arrangement with the conductive material and/or a further conductive material and thereby setting a predetermined diameter, such that the nanopore is formed. In this case, filling the fixed porous arrangement includes arranging the electrically conductive material in the fixed porous arrangement, e.g., by coating the fixed porous arrangement with the electrically conductive material or causing the latter to flow around the fixed porous arrangement, thereby narrowing a pore or pores of the fixed porous arrangement.

In this case, the nanopore may be delimited by at least two nonconductive nanoparticles in such a way that at least two nonconductive nanoparticles space apart the electrically conductive material arranged on the first electrode from the delimiting component or a conductive connection to the delimiting component. A nanopore suitable for a tunneling current measurement arises as a result.

A nanopore likewise may have a diameter of 1 to 10 nanometers, 1 to 5 nanometers, or 1 to 2 nanometers. The setting act allows a fine adjustment of the pore diameter and makes it possible to produce a nanopore having a predetermined diameter. “Customized” nanopores may thus be produced for different applications. In addition, the probability of a nanopore arising that is suitable for a tunneling current measurement, for example, is increased.

By the method, a plurality of fixed porous arrangements with nanopores may also be produced simultaneously, e.g., on a microchip.

In this case, in one embodiment, a further electrode is provided in the chamber as the delimiting component. This makes it possible, e.g., to apply a current flow from the first fixed electrode to the delimiting component. The prerequisite for measuring a tunneling current is thus provided in the fixed porous arrangement.

The coating act may include coating the electrode and the delimiting component. The coating may thus grow into the interspace from both sides.

In the interspace, in a further embodiment of the method, at least one further, conductive nanoparticle may be provided. Coating the electrode and/or filling the fixed porous arrangement with the conductive material and/or a further conductive material may include at least partly coating the at least one conductive nanoparticle. The conductive nanoparticles thus act in a shaping fashion for the coating.

In one embodiment, filling the fixed porous arrangement with the conductive material and/or a further conductive material is carried out by coating a surface delimiting the pore. In other words, a pore-delimiting wall of a fixed porous arrangement may be coated. This enables a fine adjustment of the pore diameter. The coating act and/or the filling act may be carried out by plating, in particular, by electroplating.

In an alternative or additional embodiment, filling the fixed porous arrangement includes closing the pore by coating the surface delimiting the pore with a conductive material and subsequently forming the nanopore having the predetermined diameter by removing conductive material from the closed pore. As a result, a predetermined pore diameter may likewise be set exactly. In this case, forming the nanopore may be carried out by electromigration, (e.g., by pulsed electromigration), and/or burn-through of the closed coating. Forming the nanopore by electromigration, (e.g., by pulsed electromigration), avoids high evolution of heat and makes it possible to form a nanopore that is as small as possible with a diameter of up to 1 to 2 nanometers.

Measuring the diameter of the pore, (e.g., by measuring the ion conductivity of the pore), may be carried out during the process of setting the diameter. This allows the pore diameter to be set in a controlled fashion.

In a further embodiment, the method may include guiding a biopolymer through the fixed porous arrangement and measuring a tunneling current in the nanopore for checking the presence of the nanopore. In this case, the biopolymer may be a biopolymer having a known sequence.

The object stated above is likewise achieved by a fixed porous arrangement including at least one nanopore for sequencing a biopolymer, produced by one embodiment of the method.

The object is likewise achieved by a method for sequencing a biopolymer in an apparatus, including: (1) providing at least one fixed porous arrangement in a chamber of the apparatus; (2) providing the biopolymer, in particular, a nucleic acid or a protein; (3) guiding the biopolymer through the at least one nanopore; (4) measuring a tunneling current in the at least one nanopore; and (5) determining the sequence of the biopolymer.

In this case, providing at least one fixed porous arrangement may include producing the nanopore in accordance with one embodiment of the production method. This enables the production of the fixed porous arrangement and the sequencing in the same apparatus, such that only one appliance is required for both methods.

In this example, measuring a tunneling current is carried out with the aid of a CMOS sensor (“Complementary Metal Oxide Semiconductor”) arranged in the chamber, or an electronic CMOS circuit.

A corresponding apparatus for sequencing a biopolymer is designed to carry out a method, and includes the at least one fixed porous arrangement. By virtue of the fact that the production process may be carried out in, e.g., a sequencing appliance, a nanopore (or a plurality of nanopores) may be produced directly before the sequencing operation, such that, e.g., a chip with one or a plurality of nanopore arrangements need not be stored for a long time.

### BRIEF DESCRIPTION OF THE DRAWINGS

Specific exemplary embodiments are explained below with reference to the accompanying drawings. Functionally identical elements bear the same reference signs in the figures, in which:

FIG. 1 depicts a schematic drawing of an example of a fixed porous arrangement.

FIG. 2 depicts a flow diagram of a method for producing a nanopore in accordance with one embodiment and an embodiment of a method for sequencing a biopolymer.

FIG. 3 depicts a flow diagram concerning a further embodiment of the method for producing a nanopore and a further embodiment of the method for sequencing a biopolymer.

FIG. 4 depicts a schematic illustration of the process of filling the fixed porous arrangement in accordance with an alternative embodiment.

FIG. 5 depicts a schematic elucidation of one embodiment of measuring a tunneling current in a nanopore and determining the sequence of the biopolymer.

FIG. 6 depicts an example of a diagram of the measurement currents against time.

### DETAILED DESCRIPTION

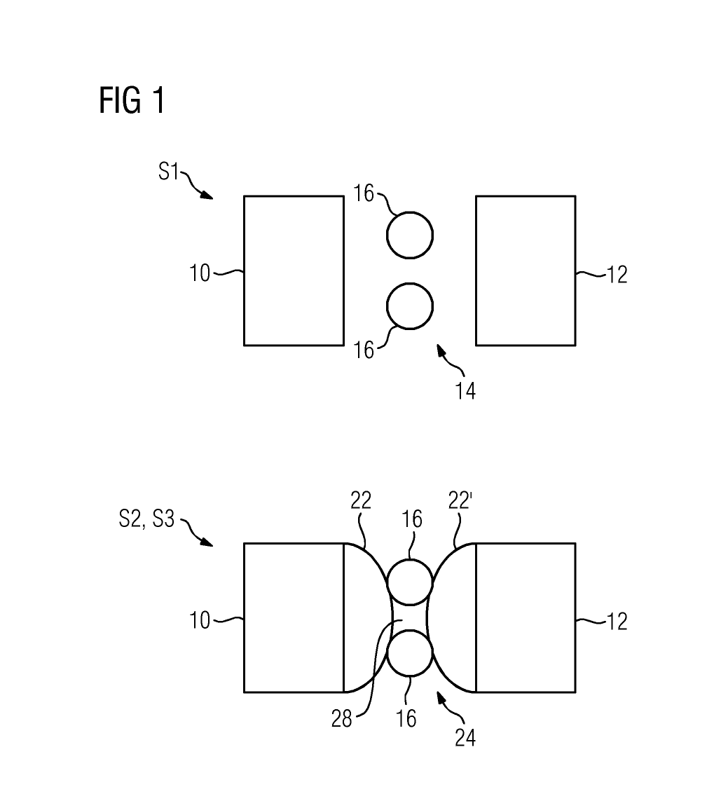

FIG. 1 schematically depicts the basic construction of a fixed porous arrangement including a nanopore 28 and the principle of the method in a simplified illustration. In an interspace 14 delimited by a first electrode 10 and a delimiting component 12, at least two nanoparticles 16 are provided in method act S1.

There is carried out a process of coating (S2) the first electrode and, here in the example, likewise the delimiting component 12 with a conductive material, that is to say, a material that includes platinum or gold, for example. There is likewise carried out a process of filling the fixed porous arrangement 24, such that a nanopore 28 arises. In the example in FIG. 1, the nanopore 28 is delimited by, for example, two nonconductive nanoparticles in such a way that the, e.g., two nonconductive nanoparticles space apart the electrically conductive material of a coating 22, (the electrically conductive material being arranged on the first electrode), from the delimiting component and a conductive connection to the delimiting component (12), (here the coating 22′), which may likewise include a conductive material. As a result, in the example of FIG. 1, a nanopore 28 arises that is suitable for a tunneling current measurement since the two electrically conductive coatings 22, 22′ depicted in FIG. 1 do not touch and a short circuit thus cannot arise.

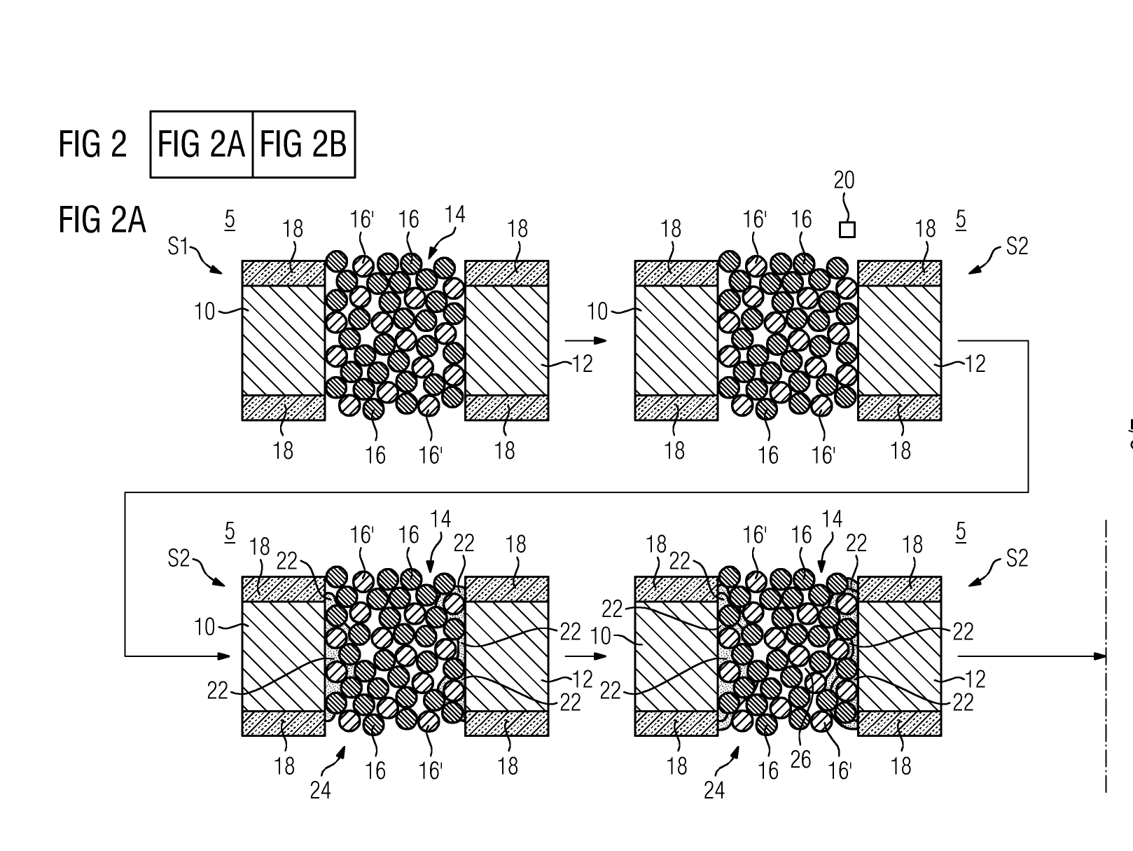

FIG. 2 illustrates one embodiment of the method for producing a nanopore. The method may be carried out for example in a chamber 5, e.g., of a sequencing appliance or of some other appliance that includes the components required for carrying out the method.

In this case, FIG. 2 depicts method act S1, in which a first electrode 10 and at least two nanoparticles 16, 16′ are provided in the chamber 5. Providing the nanoparticles 16, 16′ may be carried out, e.g., by spin-coating. In this case, the at least two nanoparticles 16, 16′ are arranged in an interspace 14 between a first electrode 10 and a delimiting component 12 situated opposite the first electrode 10, the delimiting component including a second electrode 12 in the present example. Alternatively, the interspace 14 may also be delimited by the first electrode 10 and, e.g., a wall of a conductive or insulating component 12. The electrode 10 and/or the delimiting component 12 may be arranged, as depicted in FIG. 2, between insulating layers 18 including an insulating substance, such as ceramic, glass, or silicon oxide. The arrangement of the electrode 10 and the delimiting component 12 may be fitted, e.g., on a carrier such as on a silicon wafer. Alternatively, the carrier may include a sensor, in particular, a CMOS chip or an electronic CMOS circuit.

In the present example, a multiplicity of nanoparticles 16, 16′ are provided, wherein the nanoparticles 16, 16′ include both nonconductive nanoparticles 16 and conductive nanoparticles 16′ (for the sake of clarity, only some of the nanoparticles 16, 16′ are identified by reference signs in FIG. 2 to FIG. 5). The nanoparticles have, for example, a diameter of 1 to 100 nanometers, 10 to 50 nanometers, 50 to 100 nanometers, or 1 to 10 nanometers. Nanoparticles having a diameter of 0.1 to 1 nanometer may also be used.

Method act S1 is followed by method act S2, in which the first electrode 10 and/or the delimiting component 12 are/is coated with a conductive material. Coating may be carried out, e.g., by electrochemical deposition of the conductive material, that is to say, e.g., by chemical plating by a potential difference or reducing agent, chromate treatment, electrolytic plating, or some other plating method.

By way of example, both electrodes 10, 12 are negatively polarized. After the electrode 10 has been contacted with, e.g., a corresponding plating solution, e.g., a gold complex solution such as a gold cyanide solution (a solution including Au(CN)₂, of which an Au(CN)₂ molecule is shown by way of example), after a first time interval, e.g., gold atoms of the solution deposit on the electrode 10 and a coating 22 forms on the electrode 10. In this case, the coating 22 is illustrated in cross section and in a dotted manner in FIGS. 2 to 4 and an, e.g., gold particle is illustrated as coating particle 20. Additionally or alternatively, the delimiting component 12 may also be coated. In this case, the coating 22 may reshape one or a plurality of nanoparticles 16, 16′. In this case, a conductive nanoparticle 16′ may likewise be coated, such that the coating 22 also “grows” around the conductive nanoparticle 16.

The nanoparticles 16, 16′ may thus be mechanically fixed to the coating 22 composed of the conductive material and a fixed porous arrangement 24 arises that is formed from the nanoparticles 16, 16′ and the electrically conductive coating 22.

FIG. 2 depicts that method act S2, depending on the duration of the coating process, may produce a plurality of coatings 22. After a further time interval, for example, further coating particles 20, (e.g., gold particles), are deposited on the electrodes 10, 12. The interspaces between the nanoparticles 16, 16′ are filled as a result.

FIG. 2 depicts the fixed porous arrangement 24 after a third time interval, for example, in which the plating is continued, e.g., by contacting the electrodes 10, 12 and the nanoparticles 16, 16′ with the coating particles 20. A plurality of coatings 22 are thus applied, such that, e.g., the metal “fronts” formed by the surface of the coating 22 and/or the conductive nanoparticles 16′ experience a stochastic form of propagation.

The act of the coating process does not fill the entire interspace 14 with the conductive material, and so the fixed porous arrangement 24 has at least one pore 26 (for the sake of clarity, only a few pores 26 in each case are identified by the reference sign in FIGS. 2 to 4).

The illustration at the top left in FIG. 2B reveals that a nanopore 28 is already present after the coating act. Adjacent to the nanopore 28 there is a larger pore 26 of the fixed porous arrangement 24. The method provides for setting the diameter of the pore 26 after the mechanical fixing of the nanoparticles 16, 16′. For this purpose, the fixed porous arrangement 24 is filled with the electrically conductive material and/or a further electrically conductive material (S3). In this case, as early as after the fixing of the nanoparticle or nanoparticles 16, 16′, the pore 28 of the fixed porous arrangement 24 of the example in FIG. 1 may have a diameter that is suitable for measuring a tunneling current, that is to say may be suitable as a “nanopore”. In the example, the conductive nanoparticles 16′ flanking the nanopore 28 in each case have an electrical contact via the coating 22 of the respective electrode 10, 12. The conductive nanoparticles 16′ are separated from one another by two nonconductive nanoparticles 16, with the result that no short circuit arises. For example, the adjacent pore 26 has a diameter ten times higher, and the fixed porous arrangement 24 therefore might not be suitable for, e.g., sequencing nucleic acids.

Filling the fixed porous arrangement 24 (S3) may be carried out, for example, by immersing the fixed porous arrangement 24 in a solution composed of a conductive coating particle 20, e.g., composed of a metal such as a gold complex or platinum solution or a conductive polymer such as a polyaniline solution, or causing the solution to flow around the fixed porous arrangement. Coating particles 20 then adhere, e.g., to the coating 22 and/or to the nanoparticles 16, 16′. In this way, by the number of immersing or flowing operations, the number of additional coatings 22 may be metered and the diameter of the pore 26 may thus be finely adjusted. Alternatively, filling the fixed porous arrangement 24 may also be carried out by plating with, for example, one of the plating solutions mentioned above. A controlled plating operation also enables a fine adjustment of the pore diameter.

The illustration at the top right of FIG. 2B reveals that the diameter of the pore 26 after the process of filling the fixed porous arrangement 24 is very much smaller than before the setting process, that is to say is, e.g., 1.5 nanometers and thus has a nanopore 28. This illustration therefore depicts a tunnelable configuration of the nanopores 28.

One embodiment of the method for sequencing a biopolymer, that is to say, e.g., a nucleic acid such as DNA, RNA, or an oligonucleotide, or a protein or protein fragment, is depicted in the last illustration in FIG. 2. In this case, at least one nanopore 28, (e.g., in a tunnelable configuration), is produced, for example, in accordance with the method described above. In the method, the electrodes 10, 12 may be the same electrodes 10, 12 as those for producing the at least one nanopore.

The apparatus used for this purpose may include a plurality of fixed porous arrangements 24, that is to say an array for sequencing the biopolymer 30, and/or be designed to produce a plurality of nanopores 28.

For sequencing the biopolymer 30, here, e.g., a single-stranded DNA molecule, the biopolymer 30 is provided, e.g., in a DNA sample including, e.g., different single-stranded DNA strands.

The biopolymer 30 is drawn, e.g., electrophoretically in the direction E of movement, for example, by an electric field running perpendicularly to the imaginary connection between the electrode 10 and the electrode 12, that is to say through the interspace 14. In order to be able to pass through the interspace 14, the biopolymer 30 “migrates” through the nanopore 28. As soon as the biopolymer 30 enters the nanopore 28, a tunneling current P flows from, e.g., the electrode 10 to the electrode 12. In this case, however, the two electrodes 10, 12 are not polarized identically, rather the first electrode 10 is, e.g., negatively polarized, while the second electrode 12 is positively polarized. The DNA strand that is transported through the nanopore 28 generates a characteristic tunneling current for each base.

In a method for sequencing the biopolymer 30 or for producing at least one nanopore 28, the presence of a nanopore 28 may also be checked by a biopolymer 30 that has a known sequence being guided through the fixed porous arrangement 24. If a nanopore 28 was produced successfully, then the expected sequence of the characteristic tunneling currents may be measured on the basis of this biopolymer standard.

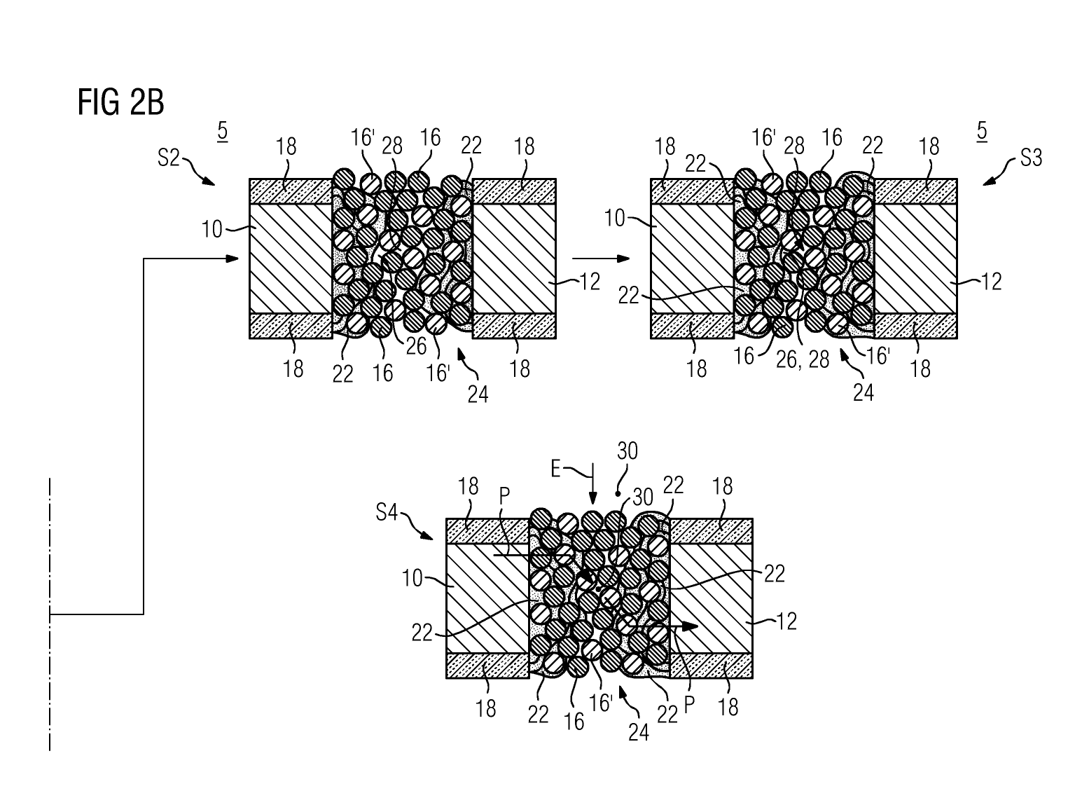

FIG. 3 depicts a method, likewise for producing a nanopore 28 in accordance with an alternative exemplary embodiment. In this case, the components and method acts identified by the corresponding reference signs correspond to those from FIG. 2 (see above). Only the differences are discussed below.

In FIG. 3, there are only nonconductive nanoparticles 16 in this example. The schematic diagram illustrates an irregular surface O of the electrodes 10, 12 that delimits the interspace 24. Such an irregular surface O, may be caused, e.g., by the process for producing the electrodes 10, 12. An arrangement depicted in the initial situation S1 makes it more difficult to measure a tunneling current P since the distance between the two conductive elements, that is to say here the electrodes 10, 12, is very large.

The process of coating the negatively polarized electrodes 10, 12 with a conductive material (S2), (e.g., platinum, palladium, or gold ions), by plating, for example, gives rise to a conductive coating 22. The conductive coating 22 reshapes the nonconductive nanoparticles 16. In this case, the irregular surfaces O predefine the shape of the coating 22, such that a bulge of the surface O brings about a bulge of the coating 22 into the interior of the interspace 24. In this case, the nonconductive nanoparticles 16 support the bulging of the coating 22.

A fixed porous arrangement 24 that arises in this case may include in this case, e.g., a plurality of pores 26, the diameter of which is above 100 nanometers, for example. As a result of the process of filling the fixed porous arrangement 24 (S3, see above) the pore 26 narrows to form a nanopore 28. With such a fixed porous arrangement 24 including a nanopore 28, it is possible to carry out a method for sequencing a biopolymer 30 as depicted in the last illustration in FIG. 3 and as was described, e.g., with regard to FIG. 2. The difference is that only the coating 22 and not the nanoparticles 16 forms an electrically conductive metal “front”.

The gap between the two electrodes 10, 12 is laterally delimited by nonconductive (that is to say insulating) nanoparticles 16 (blackened in FIG. 3), such that in contrast to the open gap the molecule to be sequenced is guided in the gap and cannot drift away. A reproducible tunneling current measurement is thus provided.

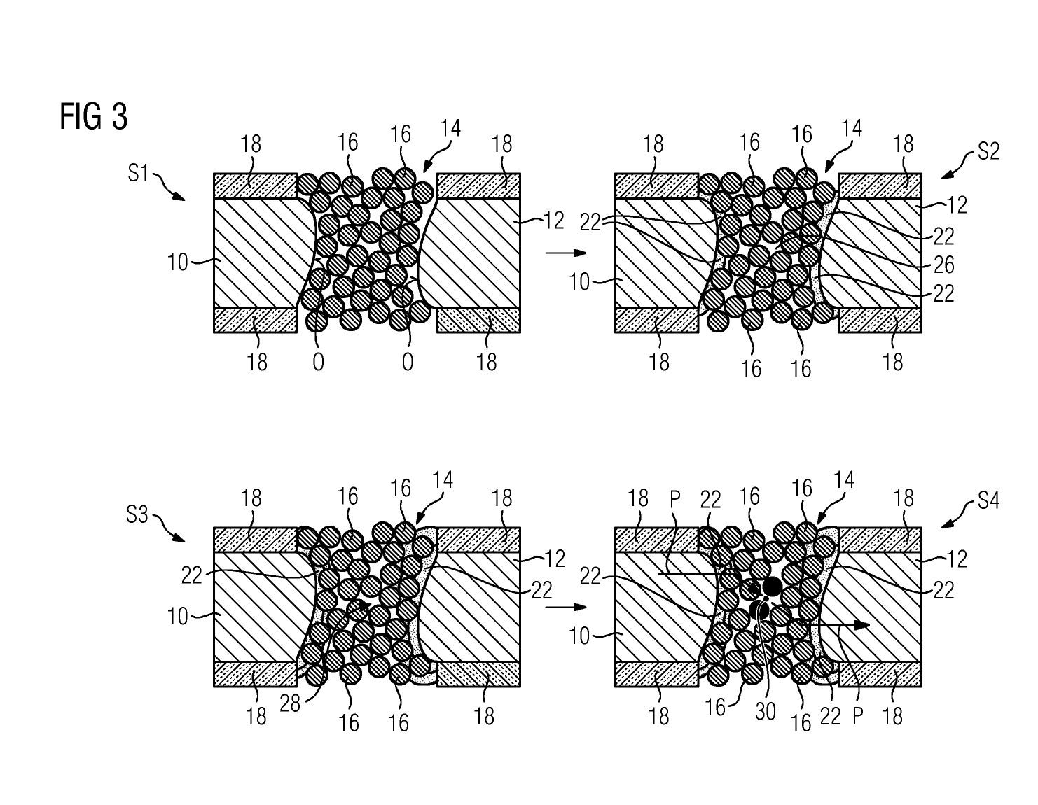

FIG. 4 depicts a further embodiment of the method for producing at least one nanopore 28. In this case, method acts S1 and S2 may be carried out in the manner as already described above.

As an alternative to the above-described variants of method act S3, setting the diameter of the pore 26 may be carried out such that the pore 26 is completely closed by the coatings 22 of the respective electrode 10, 12. In the event of the two coatings 22 touching, a short circuit occurs at the two touching points K. In the example in FIG. 4 in this case only nonconductive nanoparticles 16 are situated in the interspace 14.

Between the two electrodes 10, 12, conductive material is removed from the closed pore 26 in order to open the touching points K. For this purpose, by way of example, an electric circuit is created with the aid of a voltage source 32 and electrical lines 34. By electromigration here, for example, it is thus possible, for example, to insert (S5) a nanopore 28 having a predetermined diameter of 1 nanometer, for example. Alternatively, the touching point K may be burned through to form a nanopore 28 by a method known to the person skilled in the art. In the present example in FIG. 4, a pore 26 that does not constitute a nanopore 28 has additionally arisen in this case.

Independently of the choice of the method for coating the electrodes 10, 12 and/or for setting the pore diameter, the distance between, e.g., the coatings 22 and hence the pore diameter may be measured by, e.g., measurement of the ion conductivity of the pore 26 by a DC voltage measurement or by the measurement of an AC voltage.

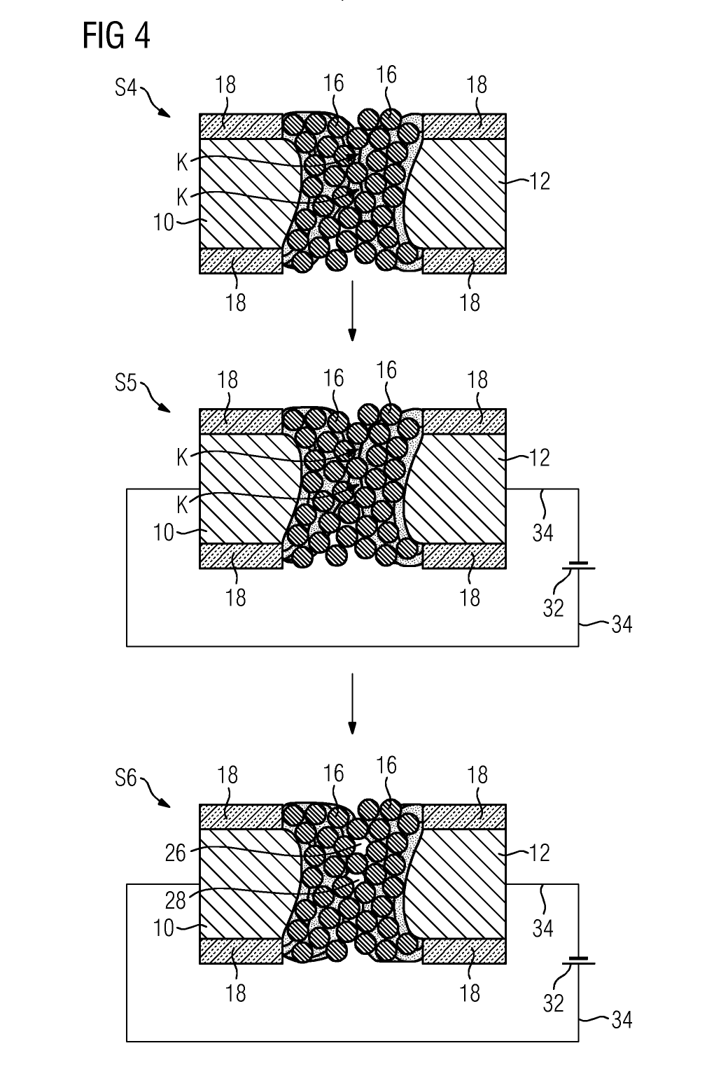

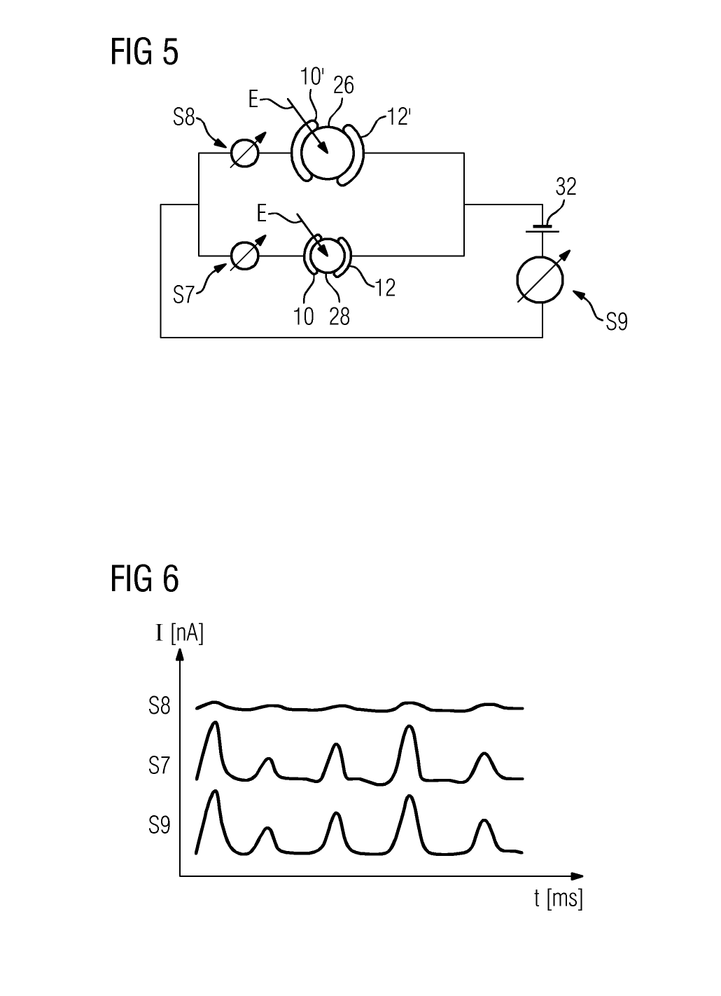

FIG. 5 depicts one example of a parallel connection of the exemplary configuration from FIG. 4, in which a large pore 26 lies adjacent to a nanopore 28. The pore 26 and the nanopore 28 are situated in a parallel connection in each case between a first electrode 10′ “lengthened” by the layer of conductive material and a second electrode 12′ “lengthened” by the layer of conductive material. In this case, a voltage source 32 generates a current that cannot flow through the pore 26 and the nanopore 28. If a biopolymer 30 enters the nanopore 28 on the path identified by the direction E of movement, a tunneling current may be measured (S7). In this case, the diagram in FIG. 6 depicts the measured current I in nanoamperes (“nA”) against a time profile t in milliseconds (“ms”) in the nanopore 28 (S7), which varies depending on, e.g., the base. The sequence of the current intensities thus corresponds, e.g., to the base sequence of the nucleic acid to be analyzed.

In the event of the biopolymer 30 entering the pore 26, no current may be measured (S8) since the diameter of the pore 26 is too large for the occurrence of the tunneling effect. Therefore, a summation signal, that is to say a total quantity of current, is measured (S9), which corresponds to the tunneling current in the nanopore 28 (S7).

The exemplary embodiments illustrate the principle of producing at least one nanopore 28 within a microstructure 24 of one or a plurality of nanoparticles 16, 16′ by, e.g., an electrodeposition of metal on at least one electrode 10, in particular, on two electrodes 10, 12.

The probability for the formation of suitable pores 28 is increased by, e.g., an electrodeposition of metal.

In accordance with one exemplary embodiment, nanobead arrangements, that is to say arrangements of nanoparticles 16, 16′, which may be unusable on account of an excessively high electrical resistance are converted into usable arrangements by metal being deposited, e.g., electrolytically on one or on both electrodes 10, 12. As a result, metal is built up on the electrode surfaces until, e.g., a closest electrically conductive nanoparticle 16′ is contacted, such that the latter also “starts to grow” (see, e.g., FIG. 2).

The deposition process may be tracked, or controlled, e.g., by electrical/electrochemical measurements. For this purpose, it is possible to measure, e.g., the ion conductivity between the electrodes 10, 12. In the event of a limit value of the conductivity being reached, for example, the plating process may be terminated.

In a further exemplary embodiment, the use of electrically conductive nanoparticles 16′ is dispensed with and only electrically insulating nanoparticles 16 are used. The deposition of the metal on, e.g., two electrodes 10, 12 is carried out non uniformly on account of uneven electrode surfaces O, or on account of obstruction by nanoparticles 16, such that the two galvanically growing metal fronts 22 stochastically approach an arbitrary point K to tunneling current distance (e.g., approximately 2 nanometers).

On account of the high packing density of electrically insulting nanoparticles 16, the gap between the two electrodes 10, 12 is laterally delimited by insulating nanoparticles 16, such that in contrast to an open gap the molecule 30 to be sequenced is guided in the gap and cannot drift away, and reproducible tunneling current measurements may thus be carried out (e.g., FIG. 2).

In one variation of the method, a “growing together” of the two electrodes 10, 12 is accepted, which may lead, e.g., to a drastic increase in the electric (in particular DC) current between the two electrodes. The plating operation is immediately terminated by a fast, sensitive control loop. The electrical short circuit produced may subsequently be “burned through,” for example, by the application of a suitably high electric current as in the case of a fuse or may be opened by electromigration and may lead to a desired nanopore junction given suitable boundary conditions. Alternatively, or additionally, suitable reagents may be used, which help to relieve the “constriction” of the short circuit, e.g., chemically (possibly during suitable electrical polarization of the electrodes 10, 12).

A better control of the translocation speed of the, e.g., nucleic acid or protein strands may be expected as a result of a packing of nanoparticles 16, 16′. In the case of “free-standing” individual nanopores 28, the translocation speed may be much too high, whereas the packings of nanoparticles 16, 16′ decelerate the polymer molecules and the speed is thus reduced.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for producing at least one nanopore having a predetermined diameter for sequencing a biopolymer in a chamber of an apparatus, the method comprising:
providing an electrode and at least two nanoparticles in the chamber, wherein the at least two nanoparticles are arranged in an interspace between the electrode and a delimiting component situated opposite the electrode,
coating the electrode with at least one electrically conductive material, thereby mechanically fixing the at least two nanoparticles in the interspace, such that a fixed porous arrangement arises in the interspace; and,
filling the fixed porous arrangement with the at least one electrically conductive material, thereby setting a diameter of at least one pore of the fixed porous arrangement to a predetermined diameter, such that the nanopore is formed, and/or wherein the nanopore is delimited by the at least two nanoparticles such that the at least two nanoparticles space apart the at least one electrically conductive material arranged on the electrode from the delimiting component or a conductive connection to the delimiting component.

2. The method as claimed in claim 1, wherein, in the chamber, a further electrode is provided as the delimiting component.

3. The method as claimed in claim 1, wherein the coating further comprises coating the delimiting component.

4. The method as claimed in claim 1, wherein, in the interspace,
at least one further, conductive nanoparticle is provided, or only nonconductive nanoparticles are provided, and
wherein, when the at least one further, conductive nanoparticle is provided, the coating of the electrode, the filling of the fixed porous arrangement, or both the coating of the electrode and the filling of the fixed porous arrangement comprises at least partly coating the at least one conductive nanoparticle.

5. The method as claimed in claim 1, wherein the filling of the fixed porous arrangement comprises coating a surface delimiting the pore with a conductive material.

6. The method as claimed in claim 1, wherein the coating of the electrode, the filling of the fixed porous arrangement, or both the coating and the filling are carried out by plating.

7. The method as claimed in claim 1, wherein the filling of the fixed porous arrangement comprises closing the pore by coating the surface delimiting the pore with a conductive material and subsequently forming the nanopore having the predetermined diameter by removing at least a portion of the conductive material.

8. The method as claimed in claim 7, wherein the forming of the nanopore is carried out by electromigration, pulsed electromigration, burn-through of the closed coating, or a combination thereof.

9. The method as claimed in claim 1, further comprising:
measuring the diameter of the pore during the filling of the fixed porous arrangement.

10. The method as claimed in claim 1, further comprising:
guiding a biopolymer through the fixed porous arrangement and measuring a tunneling current in the nanopore to check for presence of the nanopore.

11. A fixed porous arrangement comprising:
at least one nanopore for sequencing a biopolymer,
wherein the at least one nanopore is formed by:
providing an electrode and at least two nanoparticles in a chamber, wherein the at least two nanoparticles are arranged in an interspace between the electrode and a delimiting component situated opposite the electrode;
coating the electrode with at least one electrically conductive material, thereby mechanically fixing the at least two nanoparticles in the interspace, such that a fixed porous arrangement arises in the interspace; and
filling the fixed porous arrangement with the at least one electrically conductive material, thereby setting a diameter of at least one pore of the fixed porous arrangement to a predetermined diameter, such that the at least one nanopore is formed, and/or wherein the at least one nanopore is delimited by the at least two nanoparticles such that the at least two nanoparticles space apart the at least one electrically conductive material arranged on the electrode from the delimiting component or a conductive connection to the delimiting component.

12. A method for sequencing a biopolymer in an apparatus, the method comprising:
providing a fixed porous arrangement the fixed porous arrangement comprising an electrode and at least two nanoparticles in a chamber, wherein the at least two nanoparticles are arranged in an interspace between the electrode and a delimiting component situated opposite the electrode, wherein the electrode is coated with at least one electrically conductive material, thereby mechanically fixing the at least two nanoparticles in the interspace;
providing the biopolymer;
guiding the biopolymer through at least one nanopore; and
measuring a tunneling current in the at least one nanopore;
determining a sequence of the biopolymer.

13. The method as claimed in claim 12, wherein the at least one nanopore is provided by filling the fixed porous arrangement with at least one electrically conductive material, thereby setting a diameter of at least one pore of the fixed porous arrangement to a predetermined diameter, such that the at least one nanopore is formed, and/or wherein the at least one nanopore is delimited by the at least two nanoparticles such that the at least two nanoparticles space apart the at least one electrically conductive material arranged on the electrode from the delimiting component or a conductive connection to the delimiting component.

14. The method as claimed in claim 13, wherein the measuring of the tunneling current is carried out with the aid of a CMOS sensor arranged in the chamber, or an electronic CMOS circuit.

15. An apparatus for sequencing a biopolymer, the apparatus comprising:
a fixed porous arrangement having at least one nanopore, the fixed porous arrangement comprising an electrode and at least two nanoparticles in a chamber, wherein the at least two nanoparticles are arranged in an interspace between the electrode and a delimiting component situated opposite the electrode, wherein the electrode is coated with at least one electrically conductive material, thereby mechanically fixing the at least two nanoparticles in the interspace,
wherein the apparatus is configured to sequence the biopolymer by:
guiding the biopolymer through the at least one nanopore of the fixed porous arrangement;
measuring a tunneling current in the at least one nanopore; and
determining a sequence of the biopolymer.

16. The method as claimed in claim 12, wherein the biopolymer is a nucleic acid or a protein.

17. The method as claimed in claim 12, wherein the measuring of the tunneling current is carried out with the aid of a CMOS sensor arranged in the chamber, or an electronic CMOS circuit.
